(19) United States
(12) Patent Application Publication
(10) Pub. No.: US 2009/0246284 A1
(43) Pub. Date: Oct. 1, 2009

(54) O-desmethylvenlafaxine Cocrystals

(75) Inventors: Sonny Sebastian, Cherthala (IN); Krishna Challa, Hyderabad (IN); Nitin Sharadchandra Pradhan, Thane (West) (IN); Jon Valgeirsson, Hafnarfjordur (IS)

Correspondence Address: CANTOR COLBURN LLP, 20 Church Street #22, Hartford, CT, 06103-1221, US

(73) Assignee: ACTAVIS GROUP PTC EHF, Hafnarfjordur (IS)

(21) Appl. No.: 12/415,052

(22) Filed: Mar. 31, 2009

(30) Foreign Application Priority Data
Apr. 1, 2008 (IN) 818/CHE/2008

(51) Int. Cl. A61K 9/14; A61K 31/191; A61P 25/24

(52) U.S. Cl. 424/489; 514/574

(57) ABSTRACT

Disclosed herein are co-crystals comprising O-desmethylvenlafaxine and succinic acid, process for the preparation, pharmaceutical compositions, and method of treating thereof.

### CROSS REFERENCE TO RELATED APPLICATION

This application claims the benefit of priority to Indian provisional application No. 818/CHE/2008, filed on Apr. 1, 2008, which is incorporated herein by reference in its entirety.

### FIELD OF THE DISCLOSURE

The present disclosure relates to crystal forms of O-desmethylvenlafaxine, process for the preparation, pharmaceutical compositions, and method of treating thereof.

### BACKGROUND

O-Desmethylvenlafaxine, chemical name (±)-1-[2-(dimethylamino)-1-(4-hydroxyphenyl)ethyl]cyclohexanol, is a major metabolite of venlafaxine and has been shown to inhibit norepinephrine and serotonin uptake. O-Desmethylvenlafaxine, which can also be referred to as desvenlafaxine or desmethylvenlafaxine, is represented by the following structural formula:

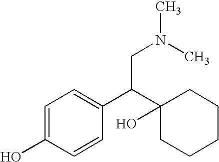

and its first synthesis was disclosed in U.S. Pat. No. 4,535,186 (hereinafter referred to as the '186 patent). Salts of O-desmethylvenlafaxine, including the fumarate, succinate, formate, maleate, tartarate and citrate salts, have been described in the literature. O-Desmethylvenlafaxine has been exemplified as a fumarate salt in the '186 patent, and succinate and formate salts were disclosed in U.S. Pat. Nos. 6,673,838 and 7,001,920, respectively. O-Desmethylvenlafaxine is also exemplified as a free base in PCT publication No. WO 00/32555.

PCT publication No. WO 2008/103461 discloses solid forms comprising stereomerically pure (−)—O-desmethylvenlafaxine, including salts thereof, particularly the hydrochloride salt, compositions comprising the solid forms, methods of making the solid forms, and methods of use thereof.

There is a need for additional crystal forms of O-Desmethylvenlafaxine.

### SUMMARY

The present inventors have now surprisingly and unexpectedly found co-crystals comprising O-desmethylvenlafaxine and succinic acid with high purity, adequate stability, good flowability and good dissolution properties.

In one aspect, provided herein is a co-crystal comprising O-desmethylvenlafaxine and succinic acid. The co-crystal can be characterized by a powder X-ray diffraction (P-XRD), Differential Scanning Calorimetry (DSC), and Scanning Electron Microscope (SEM) image.

In another aspect, provided herein is a process for the preparation of co-crystals of O-desmethylvenlafaxine and succinic acid, comprising:

a) providing a suspension of O-desmethylvenlafaxine base in an ether solvent;

b) adding succinic acid to the suspension to form a reaction mixture;

c) optionally, heating the reaction mixture; and

d) isolating a co-crystal comprising O-desmethylvenlafaxine and succinic acid from the reaction mixture.

In another aspect, provided herein is a pharmaceutical composition comprising co-crystals of O-desmethylvenlafaxine and succinic acid, and one or more pharmaceutically acceptable excipients.

In still another aspect, provided herein is a pharmaceutical composition comprising co-crystals of O-desmethylvenlafaxine and succinic acid made by the process disclosed herein, and one or more pharmaceutically acceptable excipients.

In still further aspect, encompassed herein is a process for preparing a pharmaceutical formulation comprising combining co-crystals of O-desmethylvenlafaxine and succinic acid with one or more pharmaceutically acceptable excipients.

### BRIEF DESCRIPTION OF THE DRAWINGS

FIG. 1 is a characteristic powder X-ray diffraction (XRD) pattern of co-crystals of O-desmethylvenlafaxine and succinic acid.

FIG. 2 is a characteristic differential scanning calorimetric (DSC) thermogram of co-crystals of O-desmethylvenlafaxine and succinic acid.

FIG. 3 is a characteristic Scanning Electron Microscope (SEM) image of the morphological analysis of co-crystals of O-desmethylvenlafaxine and succinic acid.

FIG. 4 is a characteristic Scanning Electron Microscope (SEM) image of the morphological analysis of O-desmethylvenlafaxine free base.

FIG. 5 is a characteristic Scanning Electron Microscope (SEM) image of the morphological analysis of succinic acid.

FIG. 6 shows the powder X-ray diffraction (XRD) patterns of cocrystals of O-desmethylvenlafaxine (ODV) and succinic acid, desmethylvenlafaxine free base and succinic acid.

### DETAILED DESCRIPTION

Co-crystallization is an established technique that is generating renewed interest in the pharmaceutical industry. Co-crystals are crystals that contain two or more non-identical molecules. Co-crystals incorporate pharmaceutically acceptable guest molecules into a crystal lattice along with the active pharmaceutical ingredient (API), changing the physical properties of the solid. Co-crystals differ from salts in the following way: in salts, a proton is transferred from the acidic to the basic functionality of the crystallization partner, as the pKa difference between the partners is sufficiently large; where as in co-crystals, no such transfer takes place.

By co-crystallizing an active agent with a guest, one can create new solid state phases, which may have improved properties over existing solid state phases of that active agent. For example, new drug formulations comprising cocrystals of active pharmaceutical ingredients (APIs) with pharmaceutically acceptable guests may have superior properties over existing drug formulations. Physical properties of active agents, or their salts, may be modified by forming a cocrystal. Such properties include melting point, density, hygroscopicity, crystal morphology, loading volume, compressibility, and shelf life.

A co-crystal can be used to isolate or purify a compound during manufacturing. If it is desirable to identify all of the solid state phases of an active pharmaceutical ingredient, then cocrystallization may be particularly desirable.

Co-crystals of O-desmethylvenlafaxine have not been described in the literature. According to one aspect, provided are co-crystals of O-desmethylvenlafaxine and succinic acid.

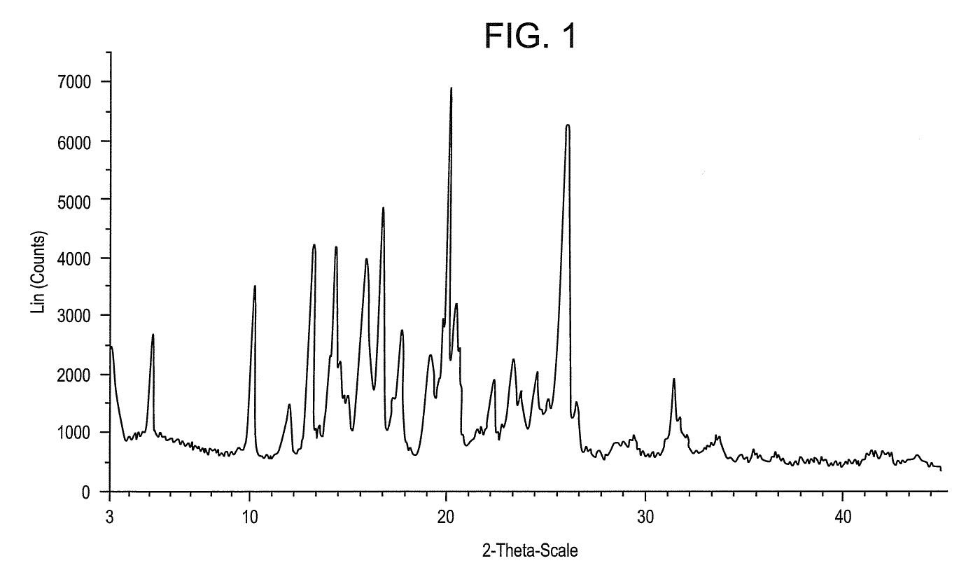

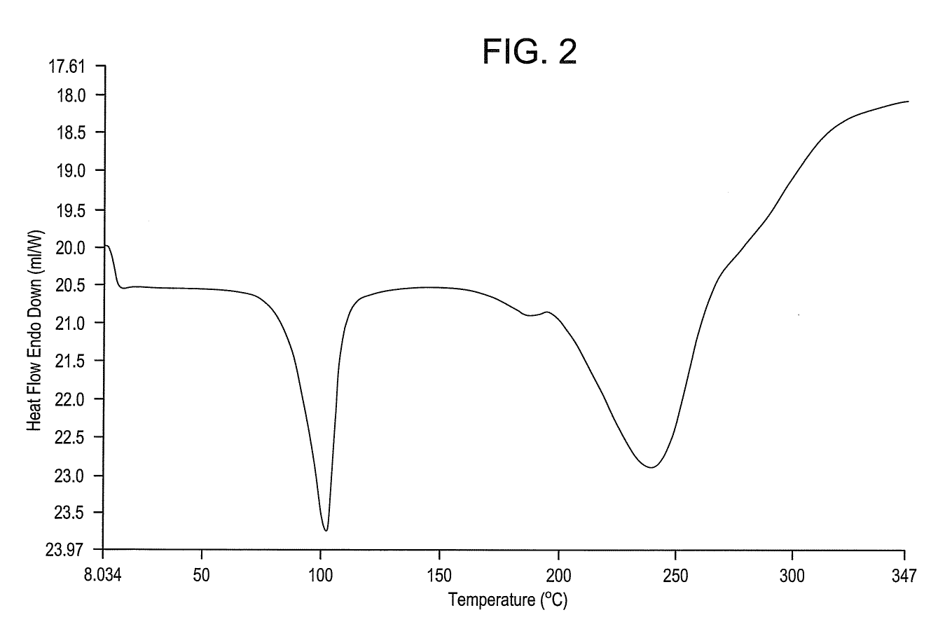

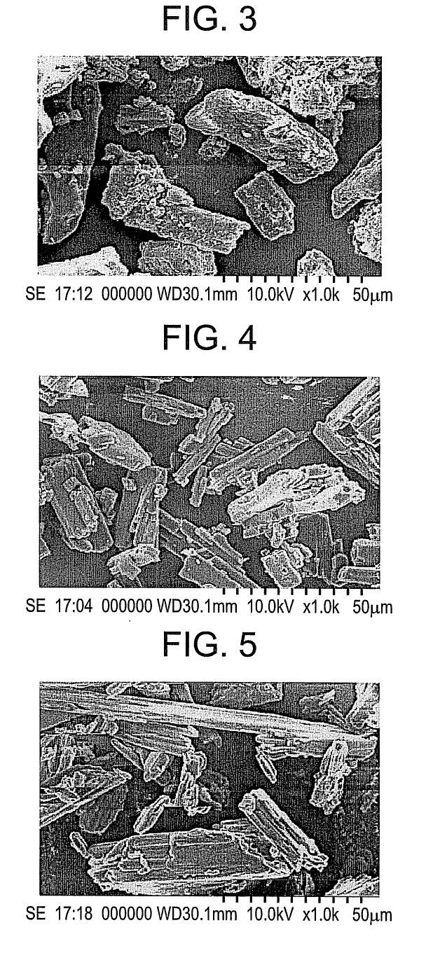

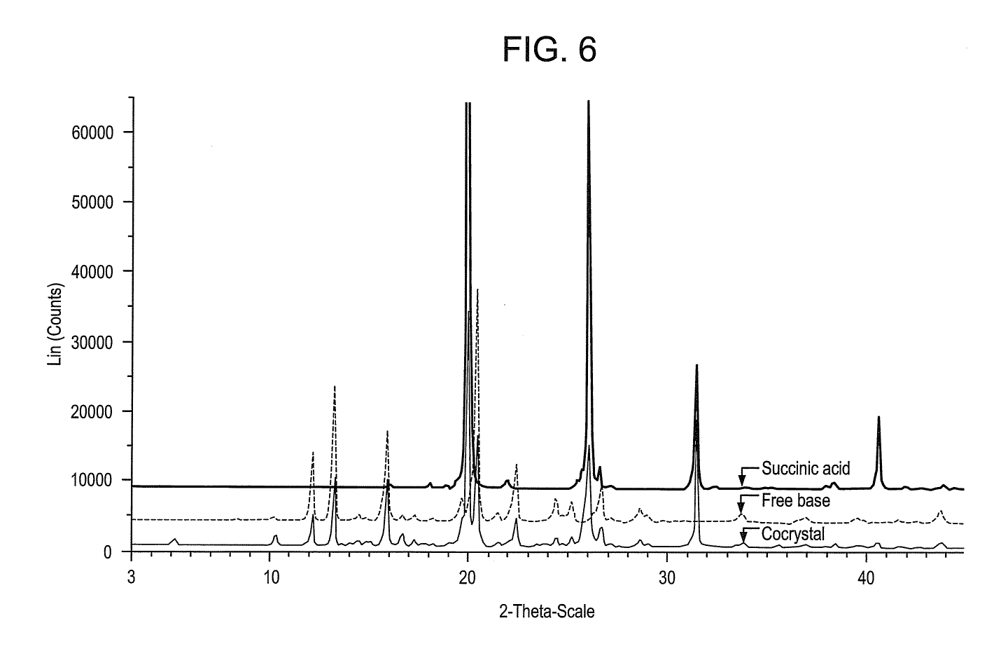

The co-crystal comprising O-desmethylvenlafaxine and succinic acid is characterized by at least one, and specifically all, of the following properties:
a) a powder X-ray diffraction pattern substantially in accordance with FIG. 1;
b) a powder X-ray diffraction pattern having peaks at about 5.08, 12.09, 15.86, 19.98, 31.48±0.2 degrees 2-theta substantially as depicted in FIG. 1;
c) a powder X-ray diffraction pattern having additional peaks at about 10.23, 13.16, 14.31, 14.57, 15.00, 15.58, 16.65, 17.27, 17.65, 19.15, 19.98, 20.40, 22.33, 23.33, 23.71, 24.50, 25.13, 25.88, 26.53, 31.73 degrees 2-theta substantially as depicted in FIG. 1; and/or
d) a differential scanning calorimetric (DSC) thermogram substantially in accordance with FIG. 2;
e) a SEM image of the morphological analysis in accordance with FIG. 3; and/or
f) a water content in the range of 1-3% by weight.

The water content of the product is determined by the Karl Fischer method.

According to another aspect, there is provided a process for the preparation of co-crystals of O-desmethylvenlafaxine and succinic acid, comprising:
a) providing a suspension of O-desmethylvenlafaxine base in an ether solvent;
b) adding succinic acid to the suspension to form a reaction mixture;
c) optionally, heating the reaction mixture obtained in step-(b); and
d) isolating co-crystals comprising O-desmethylvenlafaxine and succinic acid from the reaction mixture.

The process can produce co-crystals comprising O-desmethylvenlafaxine and succinic acid in substantially pure form.

The term “substantially pure co-crystals comprising O-desmethylvenlafaxine and succinic acid” refers to the co-crystals comprising O-desmethylvenlafaxine and succinic acid having a purity of greater than about 99 wt %, specifically greater than about 99.4 wt %, more specifically greater than about 99.5 wt % and still more specifically greater than about 99.9 wt %. The purity is preferably measured by High Performance Liquid Chromatography (HPLC).

Exemplary ether solvents used in step-(a) include, but are not limited to, diethyl ether, diisopropyl ether, methyl isopropyl ether, methyl tert.butyl ether, tetrahydrofuran, dioxane, and the like, and mixtures thereof. Specific ether solvents are diethyl ether, diisopropyl ether, methyl isopropyl ether, methyl tert.butyl ether, and mixtures thereof.

Step-(a) of providing a suspension of O-desmethylvenlafaxine base includes suspending O-desmethylvenlafaxine base in the ether solvent while stirring at a temperature below boiling temperature of the solvent used. In one embodiment, the suspension is stirred at a temperature of about 15° C. to about 110° C. for at least 30 minutes and more specifically at about 25° C. to about 80° C. for about 1 hour to about 10 hours.

Alternatively, the suspension in step-(a) is prepared by treating an acid addition salt of O-desmethylvenlafaxine with a base to liberate O-desmethylvenlafaxine base, followed by usual work up such as washings, extractions, evaporations etc., and suspending the O-desmethylvenlafaxine base in the ether solvent.

In another embodiment, the acid addition salt of O-desmethylvenlafaxine is derived from a therapeutically acceptable acid such as hydrochloric acid, hydrobromic acid, acetic acid, propionic acid, sulfuric acid, nitric acid, phosphoric acid, succinic acid, maleic acid, fumaric acid, citric acid, glutaric acid, citraconic acid, glutaconic acid, tartaric acid, malic acid, and ascorbic acid.

The treatment of an acid addition salt with base is carried out in a solvent and the selection of solvent is not critical. A wide variety of solvents such as chlorinated solvents, hydrocarbon solvents, ether solvents, alcoholic solvents, ketonic solvents, ester solvents etc., can be used.

The base can be inorganic or organic. A preferable base is an inorganic base selected from alkali metal hydroxides, carbonates and bicarbonates. A preferable alkali metal is sodium or potassium.

In one embodiment, the succinic acid used in step-(b) in the molar ratio of about 0.5 to 2.0 moles, specifically about 0.8 to 1.2 moles, and more specifically about 1 mole, per mole of O-desmethylvenlafaxine base.

In another embodiment, the addition of succinic acid in step-(b) is carried out for at least 15 minutes at below boiling temperature of the solvent used, and more specifically for about 30 minutes to about 5 hours at about 20° C. to about 40° C.

Step (c) of the process includes the optional step of heating the reaction mixture. The reaction mixture can be heated at about 35° C. to about 120° C.

The isolation co-crystals comprising O-desmethylvenlafaxine and succinic acid in step-(d) is carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method such as cooling, seeding, partial removal of the solvent from the reaction mixture, by adding an anti-solvent to the reaction mixture, or a combination thereof.

In one embodiment, the crystallization is carried out by cooling the reaction mixture at a temperature of below 30° C., and more preferably at about 0° C. to about 25° C.

The substantially pure co-crystals comprising O-desmethylvenlafaxine and succinic acid obtained in step-(d) may be recovered by methods such as filtration, filtration under vacuum, decantation, centrifugation, or a combination thereof. In one embodiment, co-crystals comprising O-desmethylvenlafaxine and succinic acid can be isolated by filtration employing a filtration media of, for example, a silica gel or celite.

The pure co-crystals comprising O-desmethylvenlafaxine and succinic acid obtained by above process may be further dried in, for example, a Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 65° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The purity of the co-crystals comprising O-desmethylvenlafaxine and succinic acid obtained by the process disclosed herein is of greater than about 99%, specifically greater than about 99.5%, more specifically greater than about 99.9%, and most specifically greater than about 99.95% as measured by HPLC. For example, the purity of the co-crystals comprising O-desmethylvenlafaxine and succinic acid disclosed herein can be about 99% to about 99.95%, or about 99.5% to about 99.99%.

Further encompassed herein is the use of the co-crystals of O-desmethylvenlafaxine and succinic acid for the manufacture of a pharmaceutical composition together with a pharmaceutically acceptable carrier.

A specific pharmaceutical composition of the co-crystals of O-desmethylvenlafaxine and succinic acid is selected from a solid dosage form and an oral suspension.

In one embodiment, the co-crystals of O-desmethylvenlafaxine and succinic acid have a D₉₀ particle size of less than or equal to about 400 microns, specifically less than or equal to about 300 microns, more specifically less than or equal to about 100 microns, still more specifically less than or equal to about 60 microns, and most specifically less than or equal to about 15 microns.

In another embodiment, the particle sizes of the co-crystals of O-desmethylvenlafaxine and succinic acid are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided pharmaceutical compositions comprising the co-crystals of O-desmethylvenlafaxine and succinic acid prepared according to processes disclosed herein and one or more pharmaceutically acceptable excipients.

According to another aspect, there is provided a process for preparing a pharmaceutical formulation comprising combining the co-crystals of O-desmethylvenlafaxine and succinic acid prepared according to processes disclosed herein, with one or more pharmaceutically acceptable excipients.

Yet in another embodiment, pharmaceutical compositions comprise at least a therapeutically effective amount of co-crystals of O-desmethylvenlafaxine and succinic acid. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The co-crystals of O-desmethylvenlafaxine and succinic acid may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain co-crystals of O-desmethylvenlafaxine and succinic acid within a capsule, which may be coated with gelatin. Tablets and powders may also be coated with an enteric coating. Suitable enteric coating include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### X-Ray Powder Diffraction (P-XRD):

The X-Ray powder diffraction was measured by an X-ray powder Diffractometer equipped with CuKα-radiations (40 kV, 40 mA) in wide-angle X-ray Diffractometer of BRUKER axs, D8 ADVANCE. The sample was analyzed using the following instrument parameters: measuring range=3-45° 2-theta; step width=0.01579°; and measuring time per step=0.11 sec.

### Differential Scanning Calorimetry (DSC):

DSC (Differential Scanning Calorimetry) measurements were performed with a Differential Scanning Calorimeter (Diamond DSC, Perkin-Elmer) at a scan rate of 5° C. per minute. The nitrogen gas purge was at 40 ml min. The instrument was calibrated for temperature and heat flow using indium as standards. The samples were encapsulated in to closed aluminium pans without hole subsequently crimped to ensure a tight seal. Data acquisition and analysis were performed using pyris software.

The following examples are provided to enable one skilled in the art to practice the invention and are merely illustrative of the invention. The examples should not be read as limiting the scope of the invention.

### EXAMPLES

### Example 1

### Preparation of Cocrystal of O-Desmethylvenlafaxine with Succinic Acid

Succinic acid (44.8 g) was added to a suspended O-desmethylvenlafaxine base (100 g) in diisopropyl ether (3.0 L) at 25-30° C. The reaction mixture was stirred for 1-2 hours at 45-50° C. The reaction mixture was slowly cooled at 25-30° C. and stirred for 4 hours. The precipitated product was filtered and dried at 45-50° C. under vacuum for 6-8 hours to produce cocrystals of O-desmethylvenlafaxine with succinic acid (Yield: 139.5 g; Water content by KF: 1.3% by weight; Purity by HPLC: 99.43%).

### Example 2

### Preparation of Cocrystal of O-Desmethylvenlafaxine and Succinic Acid

Succinic acid (4.48 g) was added to a suspended O-desmethylvenlafaxine base (10 g) in diethyl ether (100 ml) at 25-30° C. The reaction mixture was stirred for 30-45 minutes at 25-30° C. and subsequently heated at 30-35° C. for 15-20 minutes. The reaction mixture was cooled to 25-30° C. and stirred for 4 hours. The precipitated product was filtered and dried at 45-50° C. under vacuum for 18 hours to produce cocrystal of O-desmethylvenlafaxine with succinic acid (13.95 g; Water content by KF: 3.0% by weight; Purity by HPLC: 99.69%).

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles, which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent.

As used herein, D_{X} means that X percent of the particles have a diameter less than a specified diameter D. Thus, a D₉₀ or d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A co-crystal comprising O-desmethylvenlafaxine and succinic acid.

2. The co-crystal of claim 1, characterized by at least one of the following properties:
a) a powder X-ray diffraction pattern substantially in accordance with FIG. 1;
b) a powder X-ray diffraction pattern having peaks at about 5.08, 12.09, 15.86, 19.98, 31.48±0.2 degrees 2-theta substantially as depicted in FIG. 1;
c) a powder X-ray diffraction pattern having additional peaks at about 10.23, 13.16, 14.31, 14.57, 15.00, 15.58, 16.65, 17.27, 17.65, 19.15, 19.98, 20.40, 22.33, 23.33, 23.71, 24.50, 25.13, 25.88, 26.53, 31.73 degrees 2-theta substantially as depicted in FIG. 1; and/or
d) a differential scanning calorimetric (DSC) thermogram substantially in accordance with FIG. 2;
e) a SEM image of the morphological analysis in accordance with FIG. 3; and/or
f) a water content in the range of 1-3% by weight.

3. A process for the preparation of co-crystals of O-desmethylvenlafaxine and succinic acid, comprising:
a) providing a suspension of O-desmethylvenlafaxine base in an ether solvent;
b) adding succinic acid to the suspension to form a reaction mixture;
c) optionally, heating the reaction mixture obtained in step-(b); and
d) isolating co-crystals comprising O-desmethylvenlafaxine and succinic acid from the reaction mixture.

4. The process of claim 3, wherein the ether solvent is selected from the group consisting of diethyl ether, diisopropyl ether, methyl isopropyl ether, methyl tert.butyl ether, tetrahydrofuran, dioxane, and mixtures thereof.

5. The process of claim 4, wherein the ether solvent is selected from the group consisting of diethyl ether, diisopropyl ether, methyl isopropyl ether, methyl tert.butyl ether, and mixtures thereof.

6. The process of claim 3, wherein the suspension in step-(a) is provided by suspending O-desmethylvenlafaxine base in the ether solvent under stirring.

7. The process of claim 6, wherein the suspension is stirred for at least 30 minutes at a temperature below a boiling temperature of the ether solvent.

8. The process of claim 7, wherein the suspension is stirred for 1 hour to 10 hours at about 25° C. to about 80° C.

9. The process of claim 3, wherein the suspension in step-(a) is prepared by treating an acid addition salt of O-desmethylvenlafaxine with a base to produce O-desmethylvenlafaxine base and suspending the O-desmethylvenlafaxine base in the ether solvent.

10. The process of claim 3, wherein the succinic acid in step-(b) is used in a molar ratio of about 0.5 to 2.0 moles per 1 mole of O-desmethylvenlafaxine base.

11. The process of claim 10, wherein the succinic acid is used in a molar ratio of about 0.8 to 1.2 moles per 1 mole of O-desmethylvenlafaxine base.

12. The process of claim 3, wherein the addition of succinic acid in step-(b) is carried out for at least 15 minutes at a temperature below a boiling temperature of the ether solvent.

13. The process of claim 12, wherein the addition of succinic acid is carried out for 30 minutes to 5 hours at about 20° C. to about 40° C.

14. The process of claim 3, wherein the reaction mixture in step-(c) is heated at about 35° C. to about 120° C.

15. The process of claim 3, wherein the isolation in step-(d) is carried out by forcible or spontaneous crystallization.

16. The process of claim 15, wherein the forcible crystallization is initiated by cooling, seeding, partial removal of the solvent from the reaction mixture, by adding an anti-solvent to the reaction mixture or a combination thereof.

17. The process of claim 16, wherein the crystallization is carried out by cooling the reaction mixture at a temperature of below 30° C.

18. The process of claim 17, wherein the crystallization is carried out by cooling the reaction mixture at about 0° C. to about 25° C.

19. The process of claim 3, wherein the co-crystals comprising O-desmethylvenlafaxine and succinic acid obtained in step-(d) are recovered by filtration, filtration under vacuum, decantation, centrifugation, filtration employing a filtration media of a silica gel or celite, or a combination thereof.

20. The process of claim 3, wherein the co-crystals of O-desmethylvenlafaxine and succinic acid obtained in step-(d) are further dried under vacuum or at atmospheric pressure, at a temperature of about 35° C. to about 70° C.

21. The process of claim 3, wherein the co-crystals of O-desmethylvenlafaxine and succinic acid obtained have a total purity of about 99% to about 99.99% as measured by HPLC.

22. A pharmaceutical composition comprising co-crystals of O-desmethylvenlafaxine and succinic acid, and one or more pharmaceutically acceptable excipients.

23. The pharmaceutical composition of claim 22, wherein the pharmaceutical composition is a solid dosage form, an oral suspension, a liquid, a powder, an elixir, an aerosol, syrups or an injectable solution.

24. The pharmaceutical composition of claim 22, wherein the co-crystals of O-desmethylvenlafaxine and succinic acid have a D₉₀ particle size of less than or equal to about 400 microns.

25. The pharmaceutical composition of claim 24, wherein the D₉₀ particle size is less than or equal to about 300 microns; less than or equal to about 100 microns; less than or equal to about 60 microns; or less than or equal to about 15 microns.
